(19) United States
(12) Patent Application Publication
(10) Pub. No.: US 2009/0209862 A1
(43) Pub. Date: Aug. 20, 2009

(54) METHOD AND APPARATUS FOR MEDICAL ULTRASOUND DIAGNOSTICS

(75) Inventors: Balasundara Raju, Tarrytown, NY (US); Eric Cohen-Solal, Ossining, NY (US); Shervin Ayati, Carlisle, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., Eindhoven (NL)

(21) Appl. No.: 11/571,196

(22) PCT Filed: Jun. 27, 2005
(86) PCT No.: PCT/IB05/52127

§ 371 Date: Dec. 22, 2006

Related U.S. Application Data
(60) Provisional application No. 60/583,966, filed on Jun. 29, 2004.

(51) Int. Cl. A61B 8/14

(52) U.S. Cl. 600/459

(57) ABSTRACT

A method and apparatus for medical ultrasound diagnostics use time multiplexing of ultrasonic transducers of a multi-transducer array (108) disposed upon or in an application pad. Embodiments of the invention facilitate low excitation power and low wire count electrical interfaces (106, 306, 406) to the transducers and reduce electromagnetic interference between the transducers, as well as increase reliability and accuracy of positioning the application pad on the body of a patient. In one exemplary application, the invention facilitates assessment of blood perfusion.

The present invention generally relates to the medical field of ultrasonic diagnostics and, more specifically, to a method and apparatus for simplifying an ultrasound-based perfusion detecting system.

Ultrasound systems have become valuable diagnostic tools for providing, in real time, critical information about the patient's condition, such as, for example, perfusion (i.e., flow of blood), heart beat, tissue movements, and the like. Such diagnostic systems generally use non-invasive methodology based on the Doppler effect and combine high accuracy of the measurements with simplicity of diagnostic procedures.

To reduce sensitivity of the measurements to location of an ultrasonic transducer relative to a volume of interest in the body of a patient (e.g., blood vessel), an ultrasound diagnostic system typically employs an array of simultaneously activated transducers. The array may be formed on or embedded in an application pad adapted for positioning and retaining on the body. The application pad is interconnected with an electronic control unit of the system using a cable comprising pluralities of electrical wires (conductors) that, in operation, facilitate excitation of the transducers and collection of the echo signal by a data processor of the diagnostic system.

Advanced ultrasound diagnostic systems employ large arrays of simultaneously operating transducers. During the measurements, high levels of radio-frequency (RF) power used to excite multiple ultrasonic transmitters may cause parasitic cross-talks (i.e., electromagnetic interference) between the transducers. Additionally, as a number of electrical conductors in the interconnecting cable to such arrays increases, reliability and mechanical flexibility of the cable decrease. In operation, stiffness of the interconnecting cable can adversely affect positioning and retaining of the application pad on the body of a patient.

Therefore, there is a need in the art for an improved method and apparatus for ultrasound diagnostics.

The present invention is generally a method and apparatus for medical ultrasound diagnostics that use time multiplexed ultrasonic transducers. In exemplary applications, the invention facilitates detection and/or measurements of one or more of perfusion, heart beat, tissue movement, flow of a colloidal or emulsion solution, and the like.

In one aspect of the invention, the method for medical ultrasound diagnostics comprises consecutive steps of forming an array of ultrasonic transducers, periodic time multiplexing the transducers, and processing data obtained from each transducer.

In another aspect of the invention, the apparatus for medical ultrasound diagnostics comprises an array of ultrasonic transducers disposed on/in an application pad, a module for periodic time multiplexing the transducers, a control unit comprising a controller of the module, a generator for exciting the transducers, and a data processor of an echo signal, and an interconnecting cable to the control unit.

The teachings of the present invention will become apparent by considering the following detailed description in conjunction with the accompanying drawings, in which:

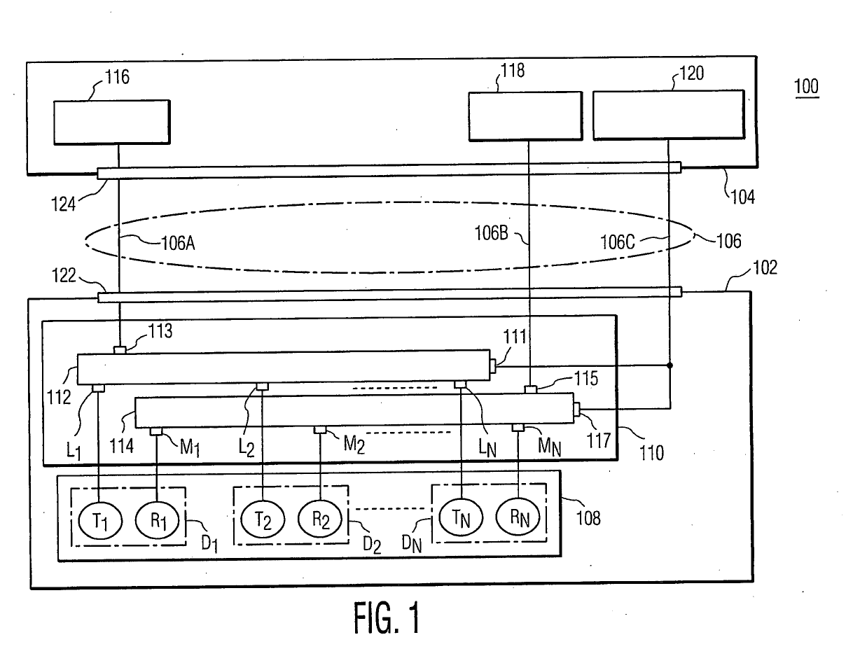

FIGS. 1-4 each depict a block diagram of an exemplary apparatus of the kind that may be used for ultrasound diagnostics in accordance with embodiments of the present invention;

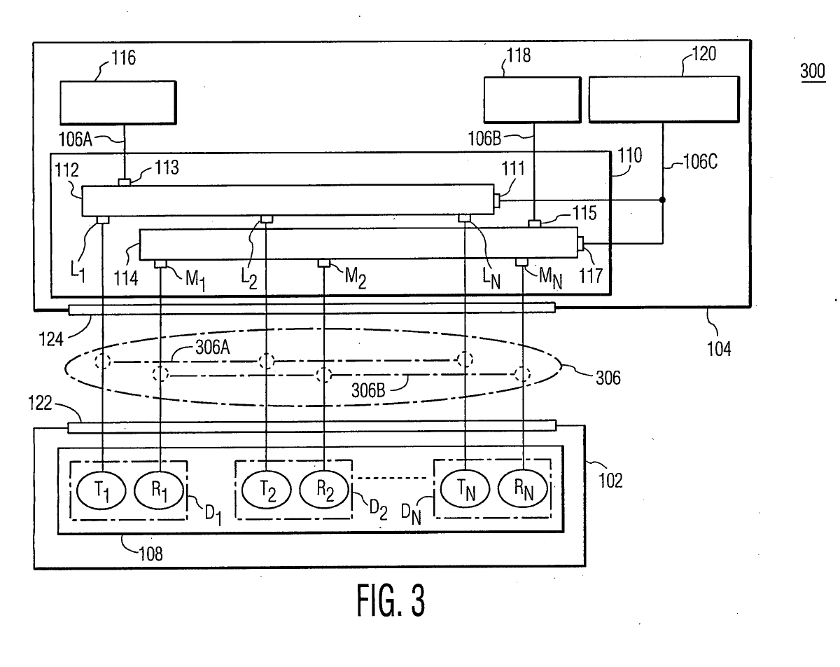

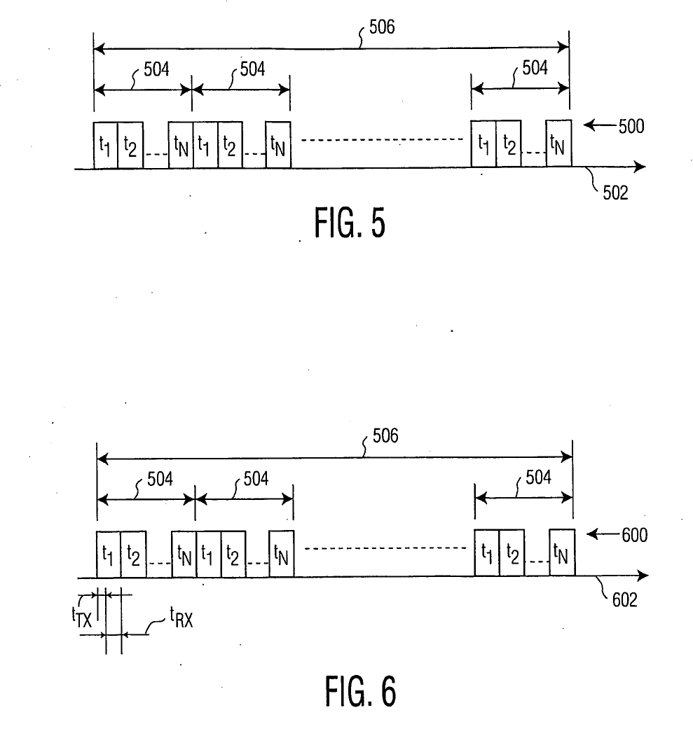

FIG. 5 depicts an exemplary timing diagram illustrating time multiplexing of ultrasonic transducers in the apparatuses of FIGS. 1 and 3;

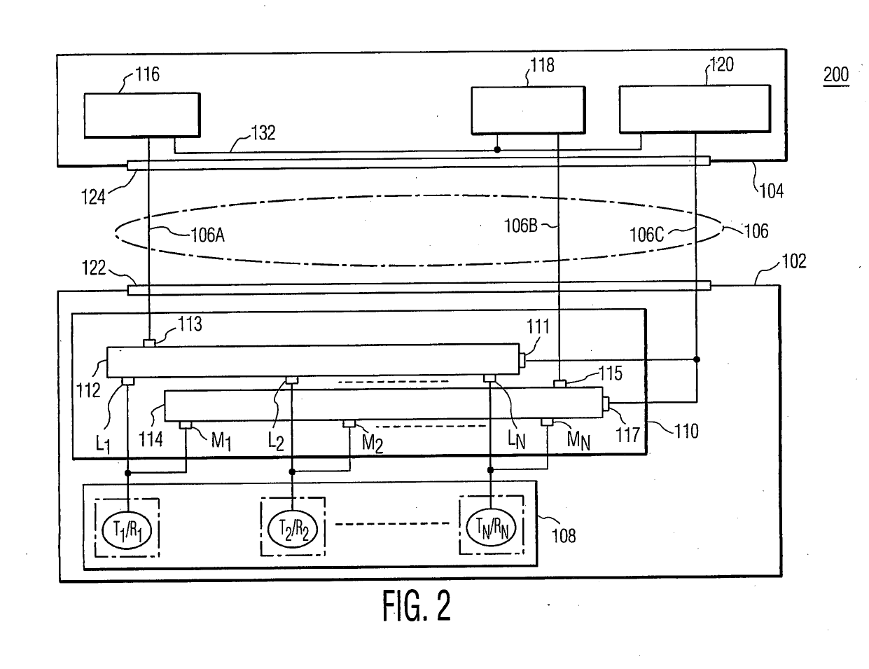

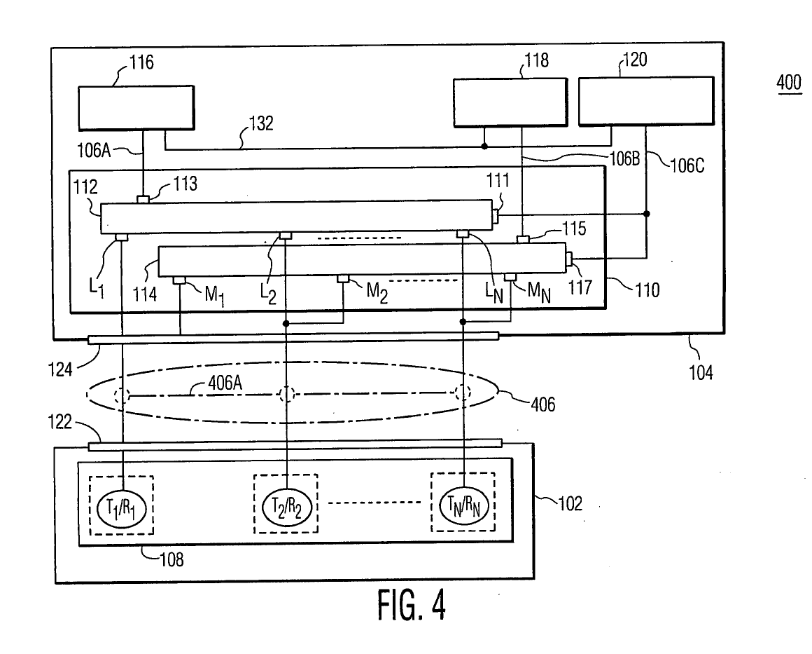

FIG. 6 depicts an exemplary timing diagram illustrating time multiplexing of ultrasonic transducers in the apparatuses of FIGS. 2 and 4, and

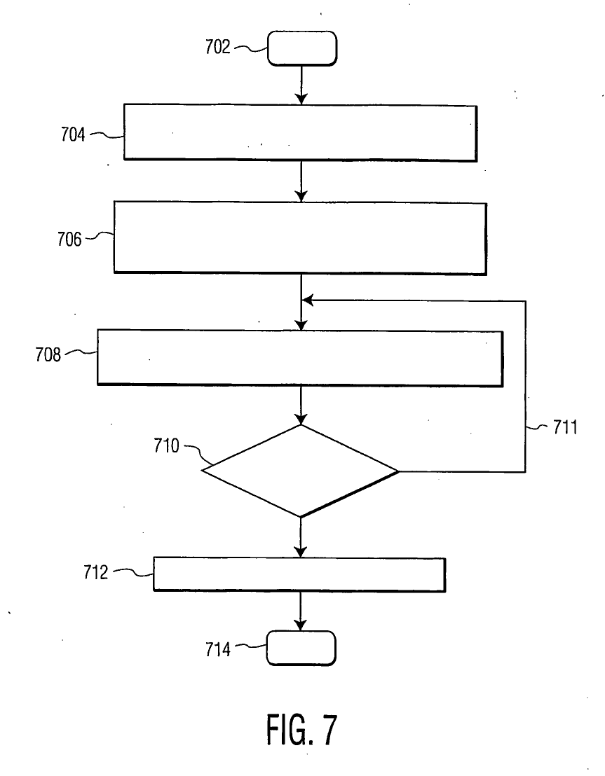

FIG. 7 depicts a flow diagram of one exemplary embodiment of the inventive method for ultrasound diagnostics that may be used during an illustrative procedure of detecting and/or measuring perfusion.

Herein, identical reference numerals are used, where possible, to designate identical elements that are common to the figures.

The appended drawings illustrate exemplary embodiments of the invention and, as such, should not be considered limiting the scope of the invention that may admit to other equally effective embodiments.

The present invention advantageously provides a method and apparatus for medical ultrasound diagnostics. Embodiments of the invention use time multiplexing of ultrasonic transducers to facilitate low wire count and low excitation power electrical interfaces to the transducers, as well as flexible mechanical interfaces between an application pad and a control unit of the apparatus.

FIG. 1 depicts a block diagram of an exemplary apparatus 100 of the kind that may be used in accordance with one embodiment of the present invention. In one exemplary application, the apparatus 100 may perform assessment (e.g., detection and/or measurements) of perfusion. Herein the term “perfusion” refers to blood flow in a blood vessel or a tissue. In other applications, the apparatus 100 may be used as a component in resuscitation systems and defibrillators, monitors and detectors of weak heart beat (e.g., fetal heart beat) or blood vessel wall movements, and the like diagnostic systems. Additionally, the apparatus 100 may also be used in non-medical devices for measuring, for example, flow of colloidal and emulsion solutions.

In one embodiment, the apparatus 100 comprises a measuring module 102, a control unit 104, and an interface 106 that interconnects the measuring module and control unit.

The measuring module 102 generally includes an array 108 of ultrasonic transducers and a multiplexing unit 110. In one embodiment, the array 108 comprises an assembly of N transducers D₁-D_{N} having transmitters T₁-T_{N} and receivers R₁-R_{N}, respectively. Herein N is an integer between, typically, 2 and 16 and, illustratively, N=4. Alternatively, the array 108 may comprise either less or more than four transducers. One such array is disclosed in commonly assigned U.S. Pat. No. 6,575,914 B2 to Rock et al. “Integrated cardiac resuscitation system with ability to detect perfusion”, which is herein incorporated by reference.

In one embodiment, the array 108 and multiplexing unit 110 are disposed on or imbedded in an application pad (not shown). The application pad may be adapted for positioning and retaining the transducers proximate a volume of interest in the body of a patient (e.g., carotid artery). The apparatus 100 may comprise a plurality of such adhering application pads each adapted for performing measurements in specific regions of the body. In an exemplary application where the apparatus 100 is used to detect and/or measure perfusion in the carotid artery, the application pad may be placed on the skin of a neck proximate to the carotid artery.

The multiplexing unit 110 facilitates selective coupling between the transducers D₁-D_{N} and components of the control unit 104. In the depicted embodiment, the unit 110 comprises multiplexers 112 and 114. In operation, the multiplexers 112 and 114 time multiplex the transmitters T₁-T_{N} (multiplexer 112) and receivers R₁-R_{N} (multiplexer 114) of the transducers D₁-D_{N}, respectively. In an alternative embodiment, the multiplexers 112 and 114 may by integrated in a single electronic device that provides multiplexing of the transmitters T₁-T_{N} and receivers R₁-R_{N} in a manner described in reference to the multiplexers 112, 114. The multiplexing unit 110 and multiplexers 112 and 114 may be implemented, for example, as electronic devices or integrated circuit (IC) electronic devices. Alternatively, the multiplexing unit 110 may be implemented as an application specific IC (ASIC).

The control unit 104 illustratively comprises a generator 116, a data processor 118, and a controller 120 of the multiplexing unit 110. In the depicted embodiment, the controller 120 is a stand-alone device. Alternatively, the controller 120 may be a portion of the data processor 118, as well as be implemented in a form of a software program executed by the data processor or a remote processor (not shown).

In one embodiment, the generator 116 is generally a source of a continuous wave (CW) radio-frequency (RF) signal (e.g., 1-10 MHz). In operation, the generator 116 is used to activate (or excite) the transmitters T₁-T_{N} of the transducers D₁-D_{N}. When excited, a transmitter generates ultrasound that propagates into the body beneath the application pad.

The data processor 118 sequentially analyzes output electrical signals from the receivers R₁-R_{N} of the transducers D₁-D_{N} and defines, e.g., perfusion in the blood vessel exposed to ultrasound generated by the transmitters T₁-T_{N}. The data processor 118 generally includes signal converters, analog and digital filters, memory devices, computer processors, and other means conventionally used for data acquisition and digital signal processing. Alternatively, portions of the digital signal processing may be performed using an external processor (not shown).

The controller 120 defines a switching state of the multiplexers 112 and 114, thus providing time multiplexing of the transducers D₁-D_{N}. In operation, the controller 120 generates and outputs a control signal that determines the configuration of conductive paths in the multiplexing unit 110. In one embodiment, the control signal is a digital code combination that configures the multiplexing module 110 to provide selective coupling between the control unit 104 and a selected transducer. When the controller 120 changes the outputted code combination, another transducer of the array 108 becomes selected. In operation, at any time only one transducer of the array 108 is coupled to the control unit 104. In particular, in the apparatus 100, the controller 120 facilitates such selective coupling between the generator 116 and a transmitter of the selected transducer and between the data processor 118 and the receiver of the same transducer, respectively. In a preferred embodiment, selective coupling is provided concurrently (i.e., simultaneously) or substantially concurrently to both the transmitter and receiver of the selected transducer.

In the depicted embodiment, transmitters T₁-T_{N} and receivers R₁-R_{N} of the transducers D₁-D_{N} are coupled to configurable (or selectable) ports L₁-L_{N} and M₁-M_{N} of the multiplexers 112 and 114, respectively. To select a port, a corresponding output signal (e.g., digital code combination) from the controller 120 may be applied to a selecting port 111 of the multiplexer 112 (ports L₁-L_{N}) or to a selecting port of the multiplexer 114, 117 (ports M₁-M_{N}). In operation, the controller 120 configures the multiplexers 112, 114 to establish electrical coupling between a transmitter (multiplexer 112) and a receiver (multiplexer 114) of the selected transducer and respective common (i.e., non-selectable) ports 113 and 115 of these multiplexers.

In operation, the multiplexers 112 and 114 concurrently couple a transmitter and a receiver of the selected transducer to the generator 116 and the data processor 118, respectively. Such concurrent coupling is provided periodically for a predetermined time interval (e.g., about 1 to 50 msec) and then terminated and is sequentially provided, one transducer at a time, for other transducers of the array 108. After all transducers of the array have been intermittently activated, another cycle of time multiplexing the transducers T₁-T_{N} begins, and these cycles are repeated until the measurements are completed. In particular, such cycles may periodically continue, e.g., for a pre-determined time interval (e.g., 2-10 sec), a multiple of duration of a cardiac cycle, or, alternatively, until a parameter of interest (e.g., perfusion) has been defined with a pre-determined degree of accuracy.

When coupled to the generator 116, a transmitter of the selected transducer generates ultrasound. Accordingly, coupling the receiver of the selected transducer to the data processor 118 facilitates acquisition, in an electrical domain, of an ultrasonic echo signal from, for example, red blood cells in blood flowing through the carotid artery. In one exemplary embodiment, duration of such intermittent coupling (i.e., time multiplexing) for each transducer D₁-D_{N} is about 10 msec. In this embodiment, ultrasound echo detected by receivers of the time multiplexed transducers may be resolved, in a frequency domain, with an error not exceeding about 100 Hz. At most diagnostic measurements, such accuracy is adequate and sufficient.

In one embodiment, the data processor 118 acquires, in real time, data from a receiver of the transducer that is currently selected (e.g., transducer D₁) and then processes the echo data during a time interval when other transducers (e.g., at least one of the transducers D₂-D_{N}) are being time multiplexed. Such a procedure is then sequentially repeated for all transducers of the array 108. Alternatively, the data processor 118 may process the data in real time, as well as after acquiring the data for a pre-determined time (e.g., a portion of a cardiac cycle). Illustratively, calculations may be performed separately for each selected transducer and further be processed (e.g., averaged) using conventional data processing techniques. To calculate the perfusion and/or related diagnostic parameter (e.g., heart beat frequency), the data processor 118 may also execute any other algorithm commonly used in the ultrasonic processing systems.

Duration of the cycle of periodic time multiplexing the array 108 is generally selected such that all transducers D₁-D_{N} may be multiplexed within a time interval equal to about 1 to 10% of duration of a cardiac cycle, while measurements of the perfusion may continue for at least duration of one cardiac cycle or, preferably, longer (e.g., 2-10 or more cardiac cycles). In one exemplary embodiment, the array 108 comprises four transducers (i.e., transducers D₁-D₄) and duration of the cycle of periodic multiplexing the transducers is about 40 msec. Such a cycle represents about 5% of duration of a typical cardiac cycle (approximately 800 msec) of a human heart.

In operation, time multiplexing of the transducers D₁-D_{N} allows to reduce output RF power of the generator 116 to a sum of the RF power that is needed to activate a single transducer and small losses of the power in the multiplexing unit 110. Time multiplexing the transducers D₁-D_{N} also increases accuracy of the echo measurements by eliminating acoustic noise from otherwise simultaneously operating transducers, as well as possible cross-talks (i.e., parasitic electrical coupling) between the transducers. Additionally, low level of RF output power results in suppression of electromagnetic interference within the apparatus 100 and between the apparatus and other electronic devices.

FIG. 2 depicts an alternate embodiment of the invention where an exemplary apparatus 200 comprises the array 108 of integrated ultrasonic transmitter/receivers T₁/R₁-T_{N}/R_{N}. Each transmitter/receiver is an ultrasonic transducer comprising a single component capable of operating as a transmitter or a receiver. In this embodiment, the generator 116 produces pulsed RF power having a duty cycle in a range of about 0.2 to 20% and a duration of an ON time interval (corresponds to a time interval t_{TX} for generating ultrasound) of about 0.2 to 20 microseconds. In the apparatus 200, the controller 120 operates the multiplexing unit 110 such that, during at least a portion of the ON time interval, a selected transducer is coupled to the generator 116 and, during at least a portion of an OFF time interval (corresponds to a time interval t_{RX} for detecting ultrasonic echo) of the duty cycle, the selected transducer is coupled to the data processor 118. Similar to the apparatus 100, such intermittent coupling is periodically provided for all transducers T₁/R₁-T_{N}/R_{N} of the array 108. Synchronization of operation of the generator 116, data processor 118, and controller 120 may be provided, for example, using a digital link 132.

In the depicted embodiment, the multiplexers 112 and 114 couple the selected transducer to the generator 116 and data processor 118, respectively. When coupled to the generator 116 during the time interval t_{TX}, the selected transmitter/receiver performs as a generator of ultrasound. Accordingly, when coupled to the data processor 118 during the time interval t_{RX}, the selected transmitter/receiver performs as a receiver of the ultrasonic echo signal. In one exemplary embodiment, in the apparatuses 100 and 200, duration of periodic intermittent coupling between the control unit 104 and each of the respective transducers D₁-D_{N} and transmitter/receivers T₁/R₁-T_{N}/R_{N}, as well as duration of time multiplexing the arrays of transducers D₁-D_{N} and transmitter/receivers T₁/R₁-T_{N}/R_{N} may generally be similar or the same. In a further embodiment, each selected transmitter/receiver T₁/R₁-T_{N}/R_{N} may be coupled to the control unit 104 during several pulse periods (t_{TX}+t_{RX}) of the generator 116.

The interface 106 generally is a cable that connects the measuring module 102 to the control unit 104. In the depicted embodiment, the cable 106 comprises branches 106A-106C and is terminated at connectors 122 and 124 of the measuring module and control unit, respectively. From a connector 122 in the measuring module 102, the branches 106A and 106B extend to the common ports 113 and 115, and the branch 106C extends to the selecting ports 111 and 117 of the multiplexers 112 and 114, respectively. Accordingly, from a connector 124 in the control unit 104, the branches 106A-106C may extend to an output terminal of the generator 116, an input terminal of the data processor 118, and an output terminal of the controller 120. In an alternate embodiment, at least one branch of the cable 106 may be terminated directly at a respective device (e.g., generator 116).

Referring to FIGS. 1 and 2, each of the branches 106A and 106B may be implemented as a single transmission line, such as a shielded wire, a twisted pair, a coaxial cable, and the like. The branch 106C may also comprise a single transmission line (i.e., serial digital bus, parallel digital bus, and the like) and, additionally, generally includes a means of a power interface for the multiplexing unit 110. In the embodiments shown in FIGS. 1 and 2, such single transmission lines provide electrical interface between the control unit 104 and the measuring module 102 having any number N (e.g., 2-16 or greater) of the transducers D₁-D_{N} (apparatus 100) or T₁/R₁-T_{N}/R_{N} (apparatus 200).

Due to low count of electrical conductors (i.e., wires) in the branches 106A-106C, the cable 106 performs as a high-reliability electrical interface, as well as a flexible mechanical interface between the control unit 104 and the application pad. Additionally, in operation, time multiplexing of the transducers D₁-D_{N} or T₁/R₁-T_{N}/R_{N} eliminates risk of cross-talks (i.e., parasitic electrical coupling) in the cable 106, thus resulting in suppression of electromagnetic interference between the transducers.

In yet further alternative embodiments shown in FIGS. 3 and 4, the multiplexing unit 110 is implemented as a portion of the control unit 104.

Referring to the embodiment shown in FIG. 3, in the apparatus 300, the measuring module 102 comprises the array 108 of the transducers D₁-D_{N} and the generator 116 operates in a CW mode. An interconnecting cable 306 between the control unit 104 and measuring unit 102 includes branches 306A and 306B that couple the multiplexers 112 and 114 to the transmitters T₁-T_{N} and receivers R₁-R_{N}, respectively. In one embodiment, each of the branches 306A and 306B comprises N transmission lines, such as shielded wires, twisted pairs, coaxial cables, and the like.

Referring to the embodiment shown in FIG. 4, in the apparatus 400, the measuring module 102 comprises the array 108 of the transmitter/receivers T₁/R₁-T_{N}/R_{N} and the generator 116 operates in a pulsed mode. In the depicted embodiment, an interconnecting cable 406 between the control unit 104 and measuring unit 102 comprises a branch 406A that couples the multiplexers 112 and 114 to the transmitter/receivers T₁/R₁-T_{N}/R_{N}. The branch 406A may comprise same transmission lines as the branch 306A or branch 306B (discussed in reference to FIG. 3 above).

In embodiments shown in FIGS. 3 and 4, time multiplexing the transducers D₁-D_{N} and T₁/R₁-T_{N}/R_{N} allows to reduce output RF power of the generator 116 to a sum of the RF power needed to activate a single transducer and small losses in the multiplexing unit 110, as well as to increase accuracy of the measurements by eliminating acoustic noise from otherwise simultaneously operating transducers.

In illustrative embodiments, the apparatuses shown in FIGS. 1-4 comprised portions of medical ultrasound systems available from Koninklijke Philips Electronics N.V. of Netherlands and multiplexing units which included multiplexing application specific integrated circuits (ASICS) or commercially available multiplexers of RF signals from Maxim Integrated Products, Inc. of Sunnyvale, Calif. (e.g., mod. MAX4708), and other suppliers.

FIG. 5 depicts an exemplary timing diagram of time multiplexing the ultrasonic transducers D₁-D_{N} in the embodiments shown in FIGS. 1 and 3. More specifically, a graph 500 depicts a sequence of cycles 504 comprising time intervals t₁-t_{N} versus time (x-axis 502). Each of the time intervals t₁-t_{N} corresponds to duration (e.g., about 10 msec) of multiplexing the respective transducer D₁-D_{N}. Time intervals t₁-t_{N} may also comprise (not shown) optional period or periods of idle time. In the depicted embodiment, during the time intervals t₁-t_{N}, the multiplexers 112 and 114 concurrently couple a transmitter and a receiver of the intermittently selected transducer to the generator 116 and the data processor 118, respectively. During each of the time intervals t₁-t_{N}, ultrasound is transmitted to the patient's body and the echo signal is received and forwarded to the data processor. The cycle 504 corresponds to duration of a period of multiplexing the transducers of the array (e.g., at N=4, about 40 msec). Together, a plurality of the cycles 504 represents duration of a time interval 506 of continuous multiplexing the transducers of the array 108. Generally, the time interval 506 relates to duration of measuring the perfusion and/or heart beat of the patient. The time interval 506 may be equal to, e.g., a multiple of duration of a cardiac cycle, a pre-determined time interval, and the like.

FIG. 6 depicts an exemplary timing diagram of time multiplexing the ultrasonic transducers T₁/R₁-T_{N}/R_{N} in the embodiments shown in FIGS. 2 and 4. In particular, a graph 600 depicts the time interval 506 and the cycles 504 (discussed in reference to FIG. 5 above) versus time (x-axis 602). Herein, each of the time intervals t₁-t_{N} corresponds to a duration (e.g., about 10 msec) of time multiplexing a respective transducer T₁R₁-T_{N}R_{N}. In the depicted embodiment, each time interval t₁-t_{N} comprises at least one pulse period of RF power that includes the time interval t_{TX} for generating ultrasound and the interval t_{RX} for receiving ultrasonic echo from the patient's body. Herein, for graphical simplicity, time intervals t_{TX} and t_{RX} are shown only for a time interval t₁ illustratively comprising only one such pulse period. The time interval t_{TX} and time interval t_{RX} corresponds to the ON and OFF interval, respectively, of the duty cycle of the generator 116. During the time intervals t_{TX} and t_{RX}, the multiplexing unit 110 couples the selected transducer to the generator 16 and data processor 118, respectively. Time intervals t₁-t_{N} may optionally be separated by periods (not shown) of idle time.

FIG. 7 depicts a flow diagram of one exemplary embodiment of the inventive method for ultrasound diagnostics. The method may be used during an illustrative procedure of detecting and/or measuring perfusion. To best understand the invention, the reader should simultaneously refer to FIGS. 1-4 and 5-6.

The method starts at step 702 and proceeds to step 704. At step 704, the application pad comprising the array 108 of the transducers D₁-D_{N} (apparatuses 100 and 300) or transducers T₁/R₁-T_{N}/R_{N} (apparatuses 200 and 400) is disposed proximate to a blood vessel (e.g., carotid artery) on the body of a patient, and then the RF generator 116, data processor 118, and controller 120 are activated.

At step 706, the controller 120 starts operating the multiplexers 112 and 114 in a manner illustrated above in FIG. 5 (apparatuses 100 and 300) or in FIG. 6 (apparatuses 200 and 400). The multiplexers facilitate periodic intermittent coupling between each transducer of the array and the generator 116 and data processor 118. During step 706, the transducers are selectively activated, one at a time. In a selected transducer, an RF output signal of the generator 116 is converted in ultrasound that propagates into the blood vessel. The transducer detects the ultrasonic echo signal from, e.g., red blood cells in the blood vessel, and converts the echo signal in an electrical format for acquiring by the data processor 118.

At step 708, the data processor 118 determines, for example, a frequency shift between the incident ultrasound and the echo signal or power of that signal to calculate the perfusion. Such calculations are generally performed for each time multiplexed transducer as relates to the time intervals t₁-t_{N} and then repeated for each cycle 504 of the time interval 506 (discussed in reference to FIGS. 5 and 6 above). In one embodiment, the data processor 118 averages the results of calculating the perfusion based upon the data acquired during each of the consecutive cycles 504. Alternatively, the data processor 118 may use other conventional techniques to increase accuracy of calculating the perfusion.

At step 710, the method queries if the data processor 110 has acquired enough echo data and completed calculations of the perfusion. If the query of step 710 is negatively answered, the method proceeds to step 708 to continue measuring perfusion, as discussed above. If the query of step 710 is affirmatively answered, the method proceeds to step 712.

At step 712, activation and time multiplexing of the transducers and, optionally, operation of the data processor 118 are terminated, and then the application pad may be removed from the body of the patient. Upon completion of step 712, the method proceeds to step 714 where the method ends.

Thus, while there have been shown and described and pointed out fundamental novel features of the present invention as applied to preferred embodiments thereof, it will be understood that various omissions and substitutions and changes in the form and details of the devices described and illustrated, and in their operation, and of the methods described may be made by those skilled in the art without departing from the spirit of the present invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is the intention, therefore, to be limited only as indicated by the scope of the claims appended hereto.

1. An apparatus for ultrasound diagnostics, comprising:
an array (108) of ultrasonic transducers disposed on or in an application pad;
a module (110) periodically time multiplexing the transducers so that signals generated by the transducers in one time period and signals received by the transducers in response to the generated signals in the one time period do not overlap with signals generated or received in another time period;
a control unit (104) comprising:
a controller (120) of the module (110);
a generator (116) for exciting the transducers; and
a data processor (118) of an echo signal detected by the transducers, and
an interconnecting cable coupling the array (108) to the control unit (104).

2. The apparatus of claim 1 wherein each transducer comprises a transmitter and a receiver.

3. The apparatus of claim 2 wherein the module (110) comprises:
a first multiplexer (112) providing intermittent coupling between the transmitter and the generator (116); and
a second multiplexer (114) providing intermittent coupling between the receiver and the data processor (118);
said intermittent couplings performed concurrently and repeated periodically for a pre-determined time.

4. The apparatus of claim 1 wherein each transducer comprises a component operating as a transmitter and a receiver.

5. The apparatus of claim 4 wherein the module comprises:
a first multiplexer (112) providing, during a first time interval, coupling between the transducer and the generator; and
a second multiplexer (114) providing, during a second time interval following the first time interval, coupling between the transducer and the data processor,
wherein the generator (116) is ON during the first time interval and OFF during the second time interval and said couplings are repeated periodically for a pre-determined time.

6. The apparatus of claim 1 wherein the module (110) is disposed on or in the application pad.

7. The apparatus of claim 6 wherein the interconnecting cable comprises a single transmission line to the generator (116) and a single transmission line to the data processor (118).

8. The apparatus of claim 1 wherein the module (110) is a portion of the control unit (104).

9. The apparatus of claim 1 wherein the generator (116) operates in a pulsed mode.

10. The apparatus of claim 9 wherein the module (110) couples a transducer to the generator when power is ON and couples the transducer to the data processor (118) when the power is OFF.

11. The apparatus of claim 1 wherein the echo signal comprises data of measuring or detecting at least one of blood perfusion, heart beat, blood vessel wall movement, and flow of a colloidal or emulsion solution.

12. The apparatus of claim 1 wherein duration of a time interval for sequential time multiplexing all transmitters comprises about 1 to 10% of duration of a cardiac cycle.

13. The apparatus of claim 1 wherein the module time multiplexes the transmitters for duration of time that is equal to or greater than duration of a cardiac cycle.

14. A method of ultrasound diagnostics, comprising:
(a) forming an array (108) of ultrasonic transducers;
(b) time multiplexing periodically the transducers of the array (108) so that signals generated by the transducers in one time period and signals received by the transducers in response to the generated signals in the one time period do not overlap with signals generated or received in another time period; and
(c) processing data obtained from the transducers.

15. The method of claim 14 wherein time multiplexing a transducer including a transmitter and a receiver is performed using concurrent intermittent coupling between the transmitter and a source of excitation and between the receiver and a processor of an echo signal.

16. The method of claim 14 wherein time multiplexing a transducer including a component operating as a transmitter and a receiver is performed using a method, comprising:
providing, during a first time interval, coupling between the transducer and a source of excitation; and
providing, during a second time interval, coupling between the transducer and a processor of an echo signal, the second time interval following the first time interval;
wherein the source of excitation is ON during the first time interval and OFF during the second time interval.

17. The method of claim 14 wherein, during the step (c), the processor processes the data sequentially obtained from each transducer.

18. The method of claim 14 wherein the data comprises results of measuring or detecting at least one of blood perfusion, heart beat, blood vessel wall movement, and flow of a colloidal or emulsion solution.

19. The method of claim 14 wherein, during the step (b), duration of a time interval for sequential time multiplexing all transmitters comprises about 1 to 10% of duration of a cardiac cycle.

20. The method of claim 14 wherein duration of the step (b) is equal to or greater than duration of a cardiac cycle.
